(19) United States
(12) Patent Application Publication
(10) Pub. No.: US 2026/0165881 A1
(43) Pub. Date: Jun. 18, 2026

(54) ULTRASONIC CAPSULORHEXIS INSTRUMENT

(71) Applicant: Alcon Inc., Fribourg (CH)

(72) Inventors: John Morgan Bourne, Irvine, CA (US); Paul R. Hallen, Colleyville, TX (US); Guangyao Jia, Irvine, CA (US); Sean Christopher Madden, Mission Viejo, CA (US); Mikhail Ovchinnikov, Dana Point, CA (US)

(21) Appl. No.: 19/416,146

(22) Filed: Dec. 11, 2025

Related U.S. Application Data
(60) Provisional application No. 63/734,294, filed on Dec. 16, 2024.

(51) Int. Cl. A61F 9/007

(52) U.S. Cl. CPC A61F 9/00745

(57) ABSTRACT

A surgical instrument includes a tube and a horn. The tube has a distal end and a proximal end. A loop is formed with a cable that is folded between a first end of the cable and a second end of the cable. The cable is at least partially disposed in the tube. The horn is configured to oscillate. The first end of the cable is coupled to the horn. An oscillation of the horn actuates the cable.

### INTRODUCTION

A capsulorhexis is a surgical procedure in which a circular portion of a lens capsule is removed to form a small hole in the anterior side of the capsule. The lens capsule surrounds the natural lens of the eye which can form a cataract after prolonged exposure to high intraocular pressure. When the cataract forms, the natural lens is removed during cataract surgery from the lens capsule and replaced with an artificial lens. The small circular hole in the lens capsule facilitates both the removal of the natural lens (e.g., emulsification) and insertion of the artificial lens.

In order to remove the portion of the lens capsule and form the small hole, a surgeon typically uses a capsulorhexis forceps which includes points/tines at distal ends of the forceps jaws. The surgeon initially pierces the lens capsule using one of the points/tines, and then grasps and repeatedly tears the lens capsule open while moving the forceps in a circular motion. Imperfections in the location, size, and/or shape of the hole formed in the lens capsule can result in surgical complications such as instability of the artificial lens and unintentional tearing of the lens capsule.

### SUMMARY

Aspects of the present disclosure relate to capsulorhexis procedures, and more specifically, to an ultrasonic instrument for performing capsulorhexis procedures.

In certain embodiments, a surgical instrument includes a tube and a horn. The tube has a distal end and a proximal end. A loop is formed with a cable that is folded between a first end of the cable and a second end of the cable. The cable is at least partially disposed in the tube. The horn is configured to oscillate. The first end of the cable is coupled to the horn. An oscillation of the horn actuates the cable.

In certain embodiments, a method includes extending a loop formed from a cable out from a distal end of a tube. The cable is at least partially disposed in the tube. The loop is disposed over a portion of a lens capsule. The portion of the lens capsule is removed by oscillating the loop at an ultrasonic frequency.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a representation of a human eye, according to embodiments described herein.

FIGS. 2A, 2B, 2C, and 2D illustrate a progression of states of extending a loop of a cable over a lens capsule, according to embodiments described herein.

FIGS. 3A, 3B, 3C, and 3D illustrate different examples of a loop of a cable, according to embodiments described herein.

FIGS. 4A, 4B, 4C, and 4D illustrate examples of a blade included in a loop, according to embodiments described herein.

FIGS. 5A and 5B illustrate examples of ultrasonic assemblies, according to embodiments described herein.

FIGS. 6A, 6B, and 6C illustrate example states of a capsulorhexis instrument, according to embodiments described herein.

FIG. 7 illustrates an example of a surgical system, according to embodiments described herein.

FIG. 8 illustrates an example method for removing a portion of a lens capsule, according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to capsulorhexis procedures, and more specifically, to an ultrasonic instrument for performing capsulorhexis procedures.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

Note that, as described herein, a distal end, segment, or portion of a component refers to the end, segment, or portion that is closer to a patient's body during use thereof. On the other hand, a proximal end, segment, or portion of the component refers to the end, segment, or portion that is distanced further away from the patient's body and is in proximity to, for example, a surgical console or a standalone light source.

Note also that, as described herein, an inferior end, segment, or portion of a component refers to the end, segment, or portion that is beneath or lower such as a bottom or underside of a tissue or structure. Conversely, a superior end, segment, or portion of the component refers to the end, segment, or portion that is above or higher such as a top or topside of the tissue or structure.

Further note that, as described herein, a medial end, segment, or portion of a component refers to the end, segment, or portion that is closest to an inside or a midline of a body. On the other hand, a lateral end, segment, or portion of the component refers to the end, segment, or portion that is closest to an outside of the body or furthest from the midline of the body.

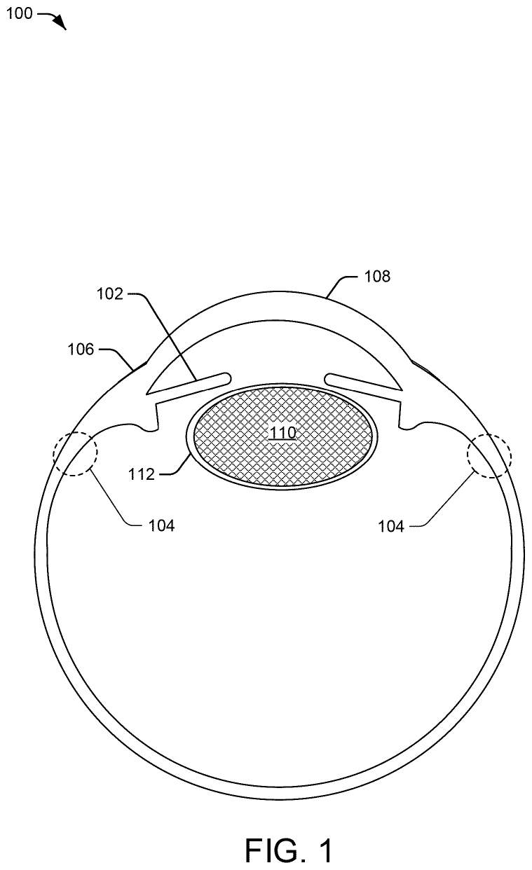

FIG. 1 illustrates a representation of a human eye 100, according to embodiments described herein. As depicted in FIG. 1, the eye 100 includes an iris 102, a pars plana 104, a sclera 106, a cornea 108, a lens 110, and a lens capsule 112.

The pars plana 104 is a region within the ciliary body commonly utilized to access the posterior segment during vitreoretinal surgical procedures (e.g., to remove portions of the vitreous humor). The sclera 106 is the white/opaque fibrous tissue that is the structural layer of the outer eye and forms its round shape. The sclera 106 extends from the cornea 108 to the optic nerve at the back of the eye. The cornea 108 covers the iris 102 which is the colored part of the eye that controls the size of the pupil. The pupil allows light into the eye 100 which the lens 110 focuses on the retina at the back of the eye. The lens 110 is contained within a lens capsule 112 which is a transparent, elastic membrane. The retina includes photoreceptor cells which convert the light into signals for visual perception.

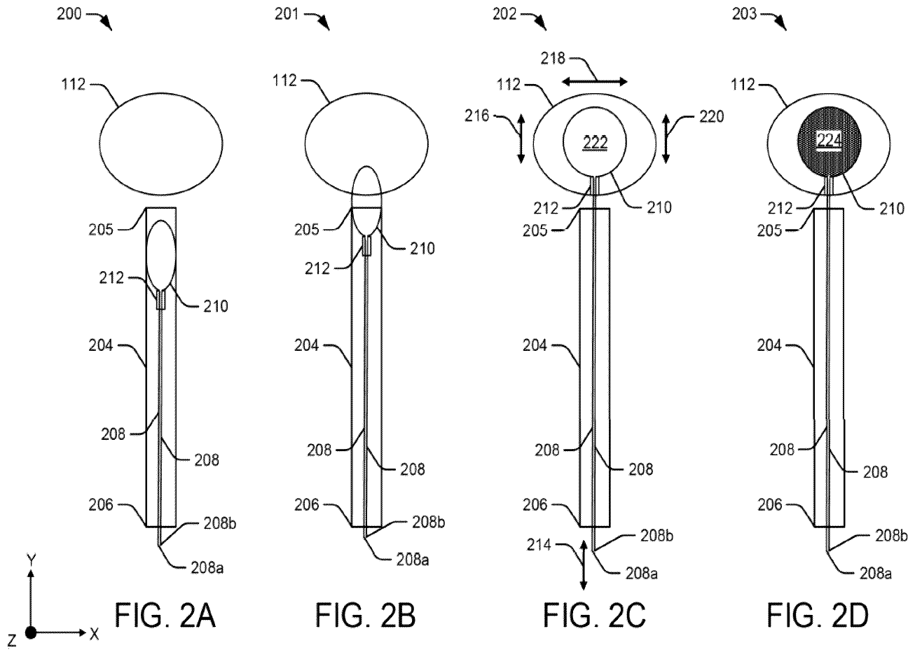

As described above, capsulorhexis is a surgical procedure in which a circular portion of the lens capsule 112 is removed to form a small hole in the anterior side of the capsule 112. FIGS. 2-7 illustrate example components of a capsulorhexis instrument for performing capsulorhexis, according to certain embodiments described herein.

FIGS. 2A, 2B, 2C, and 2D illustrate a progression of states 200-203 of extending a loop of a cable of a capsulorhexis instrument over a lens capsule 112 for performing capsulorhexis, according to embodiments described herein. As shown in FIG. 2A, in state 200, the loop 210, which is formed by a cable 208, is disposed within a tube 204 which has a distal end 205 and a proximal end 206. In the illustrated example, since the loop 210 is disposed within the tube 204, the loop 210 has a compressed shape. During the operation, the tube 204 may be inserted through an incision in the cornea 108 while the loop 210 is compressed because a decompressed loop 210 may be wider than a width of the incision.

In some embodiments, the loop 210 is formed by folding the cable 208 between a first end 208a of the cable 208 and a second end 208b of the cable 208. In state 201, which is depicted in FIG. 2B, the cable 208 is partially disposed in the tube 204 and at least one of the first end 208a or the second end 208b extends out from the proximal end 206 of the tube 204. In the embodiments of FIG. 2B, a sleeve 212 is also disposed over portions of the cable 208 adjacent to the loop 210. In some examples, the sleeve 212 may maintain the shape of the loop 210 while allowing the portions of the cable 208 disposed in the sleeve 212 to extend and retract relative to the sleeve 212.

As shown in FIG. 2C, in state 202, the loop 210 is further extended relative to the tube 204 such that no portion of the loop 210 is disposed within the tube 204 of a capsulorhexis instrument. In such a state, the loop 210 has a decompressed shape and is disposed over the lens capsule 112. For example, extending the loop 210 from the tube 204 expands the loop 210 from the compressed shape into the decompressed shape. In some embodiments, the decompressed shape of the loop 210 is circular, oval, or a similar shape. In some embodiments, the loop/cable may be made of a superelastic material with shape memory properties such as Nitinol. Other materials are also contemplated (e.g., stainless steel, polymer, etc.) In some embodiments, the loop may be coated with a polymer (e.g., a Nitinol or stainless steel loop coated with a polymer).

In some embodiments, an ultrasonic oscillation is transferred to the first end 208a of the cable 208, which causes the first end 208a to oscillate with an oscillation amplitude in directions indicated by arrows 214. As shown, the oscillations of the first end 208a are transferred to the loop 210, causing oscillations in directions indicated by arrows 216, 218, 220. The arrows 216, 220 represent actuations of the loop 210 in the Y-direction and the arrows 218 represent actuations of the loop 210 in the X-direction. The oscillations of the loop 210 then separate a portion 222 of the lens capsule 112 that is disposed within (i.e., in contact with) the loop 210 from the rest of the lens capsule 112 by cutting the lens capsule 112. In state 203, which is illustrated in FIG. 2D, the portion 222 has been removed to form a circular hole 224 in the lens capsule 112.

The circular hole 224 has an ideal size and shape for removing the lens 110 and replacing the lens 110 with an artificial lens. Forming the circular hole 224 using the oscillations of the loop 210 does not require repeated grasping and tearing of the lens capsule 112, which minimizes the risk of surgical complications. Notably, forming the circular hole 224 using the loop 210 also does not require the surgical skill necessary to perform a capsulorhexis using forceps.

In some embodiments, loop 210 may be a custom tip fitted onto a standard phacoemulsification handpiece. In some embodiments, the customized tip may include a fixed loop 210 (e.g., in the expanded configuration shown in FIG. 2D) or the customized tip may include the loop 210/tube 204 system shown in FIGS. 2A-2D with ends 208a,b attached to a distal end of the standard phacoemulsification handpiece and the loop movable relative to the tube 204. For example, the loop 210 may have a standardized threaded cone that fits on the standard phacoemulsification handpiece.

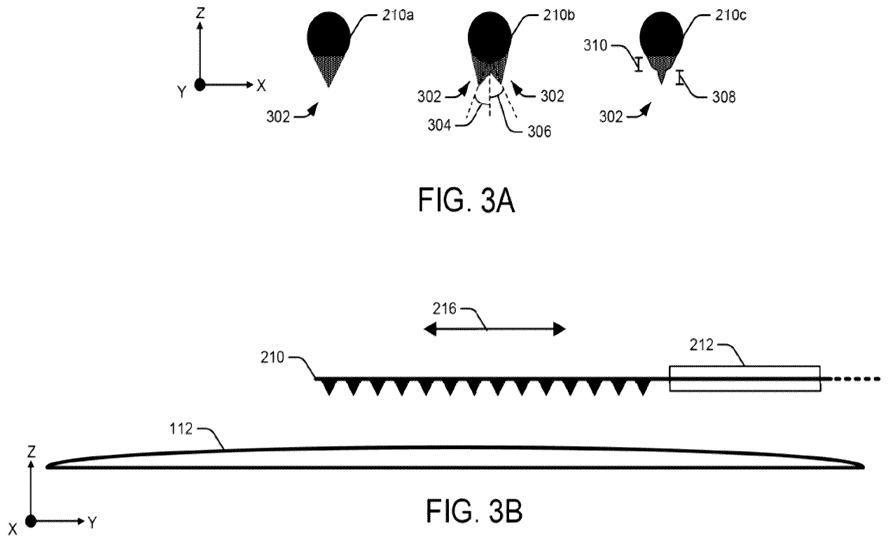

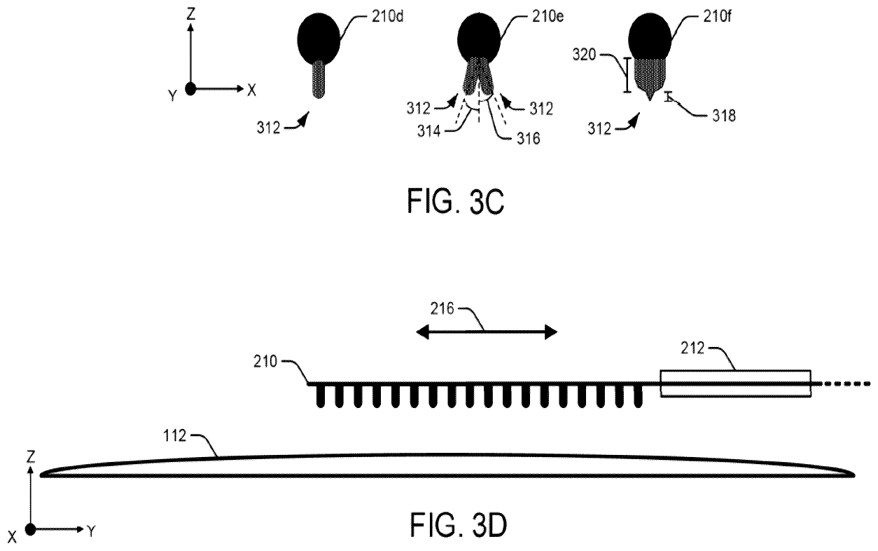

FIGS. 3A, 3B, 3C, and 3D illustrate different examples of a loop 210 of a cable 208, according to embodiments described herein. In particular, FIG. 3A illustrates front cross-sectional views of examples loops 210a, 210b, and 210c while FIG. 3B illustrates a side view of the loop 210 (which may be any of loops 210a, 210b, or 210c) adjacent to a lens capsule 112. In FIG. 3A the loops 210a-210c are presented in the X-Plane and the Y-Plane having teeth 302 that extend from an inferior end of the corresponding loop 210 in the Z-Plane. The teeth 302 are illustrated to include angled lateral edges that are separated by a greatest distance at a superior end of the teeth 302 (e.g., at the inferior end of the corresponding loop 210). The angled lateral edges of the teeth 302 meet at medial points on inferior ends of the teeth 302. In some embodiments, the dimensions of the teeth 302 are based on a thickness of the lens capsule 112. For example, the thickness of an anterior portion of the lens capsule 112 may be in a range of about 11 to 17 micrometers (μm). As such, in some embodiments, the teeth 302 extend a distance from the inferior end of the loop 210 in the Z-Plane that may be equal to or greater than the thickness of the anterior portion of the lens capsule 112. In some embodiments, the teeth 302 may extend about 20 μm from the loop 210 in the Z-Plane. In some embodiments, the loop 210 may be a Nitinol, stainless steel, or polymer loop with teeth 302 or a tapered profile or bevel to create an edge that is placed against the capsule to create the capsulorhexis.

In some embodiments, instead of or in addition to having teeth, the loop 210 may be coated with a material that increases the friction of the loop 210 against the capsule surface to make the capsulorhexis when the loop is ultrasonically vibrated. For example, the loop may be coated in a diamond dust coating or a diamond-like carbon coating. Other coating materials are also contemplated.

As shown in FIG. 3A, the teeth 302 of loops 210a-210c are oriented in the X-Plane and the Z-Plane relative to a cross-sectional view of the loop 210. In the example of loop 210a, the teeth 302 are oriented in a normal direction with respect to the cross-sectional view of the loop 210a in the Z-plane. In the example of loop 210b, the teeth 302 are oriented at angles relative to the cross-sectional view of the loop 210b in the Z-plane such that a first tooth is oriented at a first angle 304 relative to the cross-sectional view of the loop 210b in the Z-plane and a second tooth is oriented at a second angle 306 relative to the cross-sectional view of the loop 210b in Z-plane. In the example of loop 210c, the teeth 302 each have a first portion 308 and a second portion 310. In some embodiments, the first portion 308 is formed by concave lateral edges of the tooth 302 and configured to pierce the lens capsule 112 with a minimum amount of force applied to the lens capsule 112 by the loop 210c. In some embodiments, the second portion 310 is configured to increase an amount of force required to pierce the lens capsule 112 relative to the minimum amount of force used to pierce the lens capsule 112 in order to provide tactile feedback that the first portions 308 have pierced the lens capsule 112.

In some embodiments, the ultrasonic oscillation is not transferred to the first end 208a of the cable 208 until the loop 210 is disposed over the lens capsule 112 such that the inferior ends of the teeth 302 contact or depress the lens capsule 112. After disposing the loop 210 over the lens capsule 112 such that the teeth 302 contact or depress the lens capsule 112, transferring the ultrasonic oscillation to the first end 208a of the cable 208 oscillates the teeth 302 relative to the lens capsule 112. As shown in FIG. 3B, transferring the ultrasonic oscillation to the first end 208a oscillates the teeth 302 in the Y-direction relative to the lens capsule 112 which is indicated by the arrows 216. In some embodiments, oscillating the teeth 302 relative to the lens capsule 112 removes a portion (e.g., portion 222 shown in FIG. 2) of the lens capsule 112 and forms the circular hole (e.g., circular hole 224 shown in FIG. 2). Although the ultrasonic oscillation is described as being transferred to the first end 208a of the cable 208 after the loop 210 is disposed over the lens capsule 112, it is to be appreciated that, in some embodiments, the ultrasonic oscillation may be transferred to the first end 208a of the cable 208 before the loop 210 is disposed over the lens capsule 112.

FIG. 3C illustrates front cross-sectional views of examples loops 210d, 210e, and 210f while FIG. 3D illustrates a side view of the loop 210 (which may be any of loops 210d, 210e, or 210f) adjacent to a lens capsule 112. In FIG. 3C, the loops 210d-210f are presented in the X-Plane and the Y-Plane having teeth 312 that extend from an inferior end of the corresponding loop 210 in the Z-Plane. The teeth 312 are illustrated to include parallel lateral edges. The parallel lateral edges of the teeth 312 form convex edges at inferior ends of the teeth 312. In some embodiments, the dimensions of the teeth 312 are based on a thickness of the anterior portion of the lens capsule 112 which may be in a range of about 11 to 17 micrometers (μm). Accordingly, in some embodiments, the teeth 312 extend a distance from the inferior end of the loop 210 in the Z-Plane that is equal to or greater than the thickness of the anterior portion of the lens capsule 112. In some embodiments, the teeth 312 may extend about 20 μm from the loop 210 in the Z-Plane.

As shown in FIG. 3C, the teeth 312 of loops 210d-210f are oriented in the X-Plane and the Z-Plane relative to a cross-sectional view of the loop 210. In the example of loop 210d, the teeth 312 are oriented in a normal direction with respect to the cross-sectional view of the loop 210d in the Z-plane. In the example of loop 210e, the teeth 312 are oriented at angles relative to the cross-sectional view of the loop 210e in the Z-plane such that a first tooth is oriented at a first angle 314 relative to the cross-sectional view of the loop 210e in the Z-plane and a second tooth is oriented at a second angle 316 relative to the cross-sectional view of the loop 210e in Z-plane. In the example of loop 210f, the teeth 312 each have a first portion 318 and a second portion 320. In some embodiments, the first portion 318 is formed by concave lateral edges of the tooth 312 and configured to pierce the lens capsule 112 with a minimum amount of force applied to the lens capsule 112 by the loop 210f. In some embodiments, the second portion 320 is configured to increase an amount of force required to pierce the lens capsule 112 relative to the minimum amount of force used to pierce the lens capsule 112 in order to provide tactile feedback that the first portions 318 have pierced the lens capsule 112.

In some embodiments, the loop 210 is disposed over the lens capsule 112 such that the convex edges at the inferior ends of the teeth 312 contact or depress the lens capsule 112. The ultrasonic oscillation is transferred to the first end 208a of the cable 208 which oscillates the teeth 312 relative to the lens capsule 112. As shown in FIG. 3D, transferring the ultrasonic oscillation to the first end 208a oscillates the teeth 312 in the Y-direction relative to the lens capsule 112 which is indicated by the arrows 216. In some embodiments, oscillating the teeth 312 relative to the lens capsule 112 removes a portion (e.g., portion 222 shown in FIG. 2) of the lens capsule 112 and forms the circular hole (e.g., circular hole 224 shown in FIG. 2).

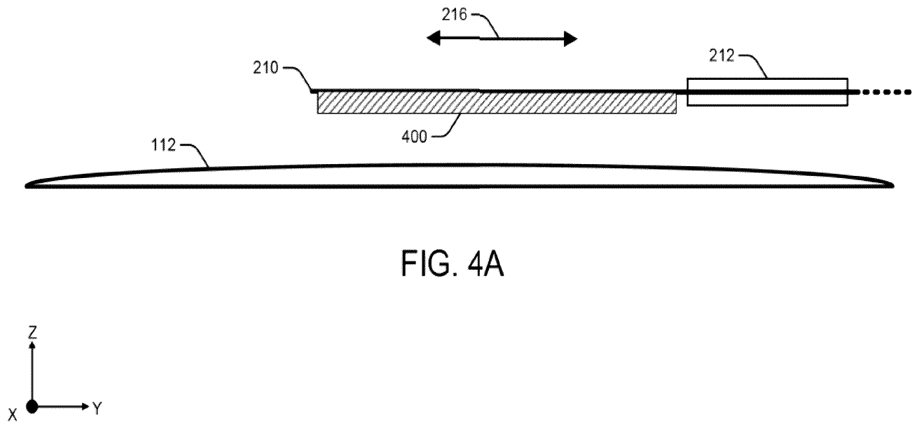

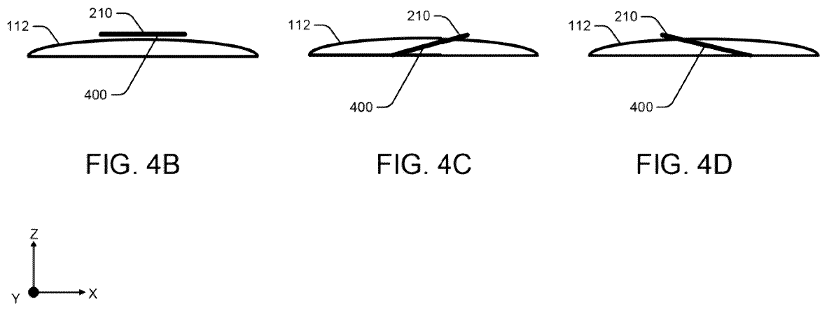

FIGS. 4A, 4B, 4C, and 4D illustrate examples of a loop 210 with a blade 400, according to embodiments described herein. FIG. 4A illustrates a side view of the loop 210 including the blade 400. In FIG. 4A, the loop 210 is shown in the X-Plane and the Y-Plane with the blade 400 extending from the loop 210 in the Z-Plane. In some embodiments, the blade 400 has dimensions based on the thickness of the lens capsule 112. For example, the blade 400 may extend about 20 μm from the loop 210 in the Z-Plane. In some embodiments, transferring the ultrasonic oscillation to the first end 208a of the cable 208 is configured to oscillate the blade 400 relative to the lens capsule 112. Oscillating the blade 400 relative to the lens capsule 112 may remove a portion (e.g., portion 222 shown in FIG. 2) of the lens capsule 112 and form the circular hole (e.g., circular hole 224 shown in FIG. 2).

FIG. 4B illustrates the loop 210 being disposed over the lens capsule 112. In FIG. 4B, the ultrasonic oscillation is not transferred to the first end 208a of the cable 208. In FIG. 4C, the ultrasonic oscillation is transferred to the first end 208a of the cable 208 and the loop 210 is illustrated at a midpoint of a first half of an example oscillation cycle. As shown, at the midpoint of the first half of the example oscillation cycle, a first portion of the blade 400 is driven through the lens capsule 112. In FIG. 4D, the loop 210 is illustrated at a midpoint of a second half of the example oscillation cycle. In some embodiments, during the second half of the example oscillation cycle, a second portion of the blade 400 is driven through the lens capsule 112. In some embodiments, the example oscillation cycle removes a portion (e.g., portion 222 shown in FIG. 2) of the lens capsule 112 and forms the circular hole (e.g., circular hole 224 shown in FIG. 2).

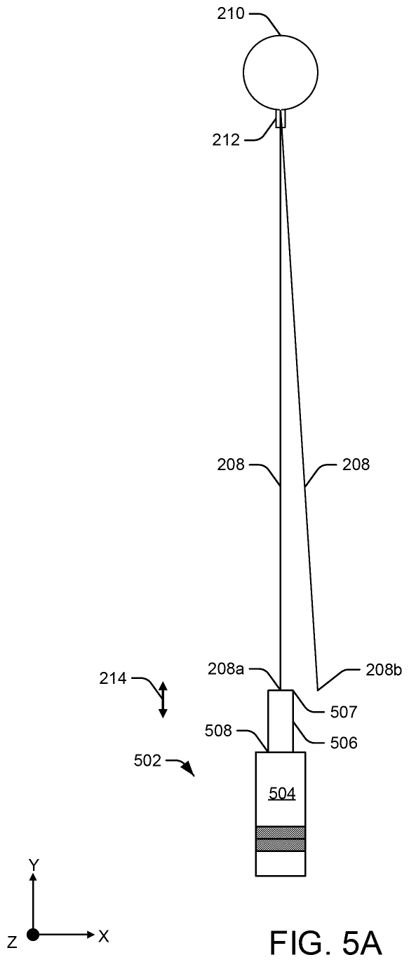

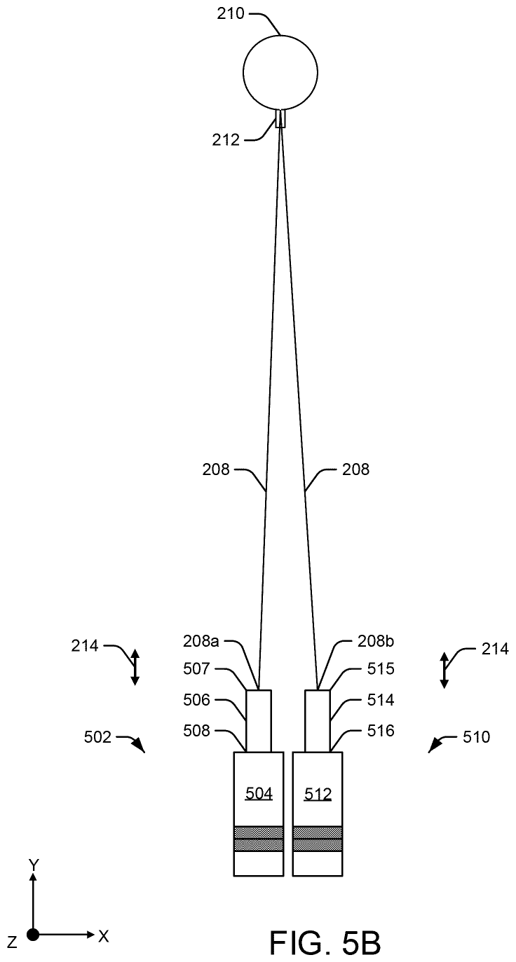

FIGS. 5A and 5B illustrate examples of ultrasonic assemblies, according to embodiments described herein. FIG. 5A illustrates a representation of an ultrasonic assembly with a first oscillator 502. The first oscillator 502 includes a transducer 504 and an ultrasonic horn 506. The ultrasonic horn 506 has a distal end 507 and a proximal end 508, and the transducer 504 is coupled to the proximal end 508 of the ultrasonic horn 506. In some embodiments, the first end 208a of the cable 208 is coupled to the distal end 507 of the ultrasonic horn 506. Energizing the transducer 504 at an ultrasonic resonance frequency causes the distal end 507 of the ultrasonic horn 506 to oscillate at a first oscillation amplitude. The first oscillation at the first oscillation amplitude is transferred to the cable 208 via the first end 208a of the cable 208. Oscillating the cable 208 extends and retracts the cable 208 which actuates the loop 210 relative to the lens capsule 112 and forms the circular hole (e.g., circular hole 224 shown in FIG. 2) by removing the portion (e.g., portion 222 shown in FIG. 2).

FIG. 5B illustrates a representation of an ultrasonic assembly with the first oscillator 502 and a second oscillator 510. The second oscillator 510 includes a transducer 512 and an ultrasonic horn 514. The ultrasonic horn 514 includes a distal end 515 and a proximal end 516. The transducer 512 is coupled to the proximal end 516 of the ultrasonic horn 514 and the second end 208b of the cable 208 is coupled to the distal end 515 of the ultrasonic horn 514. Energizing the transducer 512 at an ultrasonic resonance frequency causes the distal end 515 of the ultrasonic horn 514 to oscillate at a second oscillation amplitude. The first oscillation amplitude may be the same as or different from the second oscillation amplitude. In some embodiments, the oscillation at the second oscillation amplitude is transferred to the cable 208 via the second end 208b of the cable 208.

In various embodiments, the ultrasonic assembly shown in FIG. 5B may be configured to increase a total displacement of a portion of the loop 210 relative to the ultrasonic assembly shown in FIG. 5A. In one or more embodiments, the transducers 504, 512 may have the same resonance frequency or different resonance frequencies. In some embodiments, the first oscillator 502 and second oscillator 510 may be configured to oscillate in phase or out of phase.

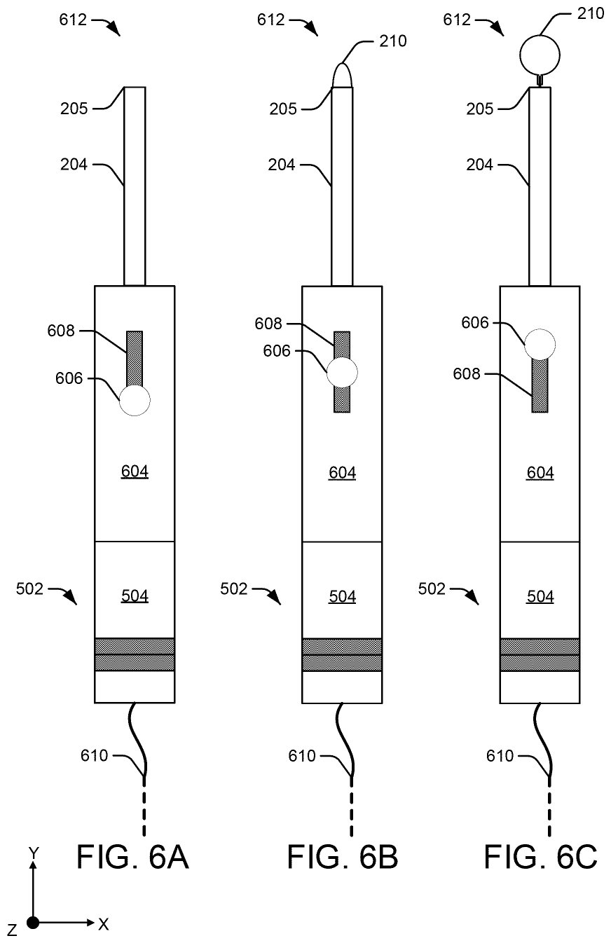

FIGS. 6A, 6B, and 6C illustrate example states of a capsulorhexis instrument 612, according to embodiments described herein. As shown in FIGS. 6A, 6B, and 6C, the first oscillator 502 is partially disposed in a handle 604 and the tube 204 is also partially disposed in the handle 604. In some embodiments, the handle 604 includes an actuator 606 that is configured to actuate within a channel 608.

In FIG. 6A, the actuator 606 is fully retracted in the channel 608 and the loop 210 is disposed within the tube 204. In some embodiments, when the actuator 606 is fully retracted in the channel 608, the distal end 205 of the tube 204 is inserted into an incision in the cornea 108. In FIG. 6B, the actuator 606 is partially extended in the channel 608 and the loop 210 partially extends from the distal end 205 of the tube 204. In FIG. 6C, the actuator 606 is fully extended in the channel 608 and the loop 210 fully extends from the distal end 205 of the tube 204. For example, the extension of the loop 210 from the tube 204 expands the loop 210 from the compressed shape into the decompressed shape. In some embodiments, when the actuator 606 is fully extended in the channel 608, the transducer 504 is energized via a cord 610 to oscillate the loop 210 relative to the lens capsule 112. In some embodiments, oscillating the loop 210 relative to the lens capsule 112 forms a circular hole (e.g., circular hole 224 shown in FIG. 2) by removing the portion (e.g., portion 222 shown in FIG. 2) of the lens capsule 112. The cord 610 may connect to an ultrasonic generator that is also used to emulsify the lens 110 or the cord 610 can connect to another ultrasonic generator.

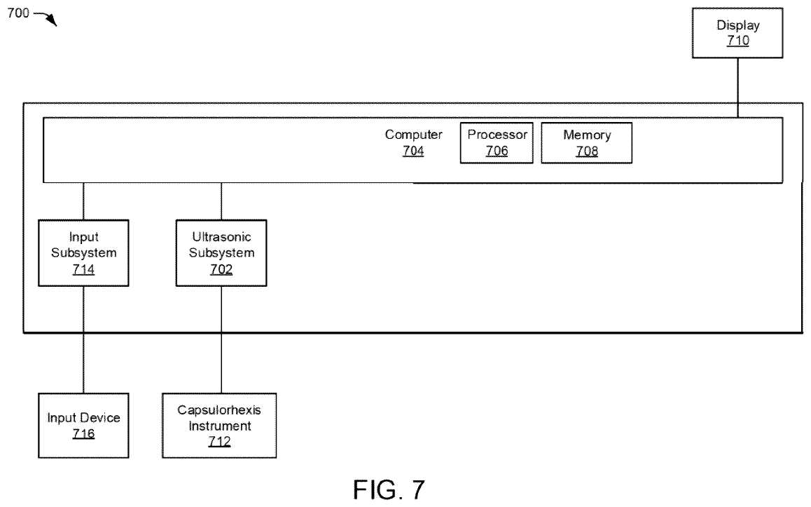

FIG. 7 illustrates an example of a surgical system 700, according to embodiments described herein. The surgical system 700 includes an ultrasonic subsystem 702 which is coupled to a computer 704. The computer 704 includes a processor 706 and a memory 708. A display device 710 is coupled to the computer 704. In some embodiments, the display device 710 includes a user interface configured to receive user inputs and communicate received user inputs to the computer 704. A capsulorhexis instrument 712 is coupled to the ultrasonic subsystem 702. One example of the capsulorhexis instrument 712 includes the capsulorhexis instrument 612 depicted in FIGS. 6A-6C. The capsulorhexis instrument 712 includes the loop 210.

An input subsystem 714 is coupled to the computer and an input device 716 such as a footswitch. In some embodiments, a user interaction with the input device 716 is communicated to the computer 704 via the input subsystem 714. The processor 706 executes instructions that cause the processor 706 to control the ultrasonic subsystem 702 and energize a transducer (e.g., transducer 504) in the capsulorhexis instrument 712. In some embodiments, energizing the transducer oscillates a loop (e.g., loop 210) of the capsulorhexis instrument 712 relative to a lens capsule.

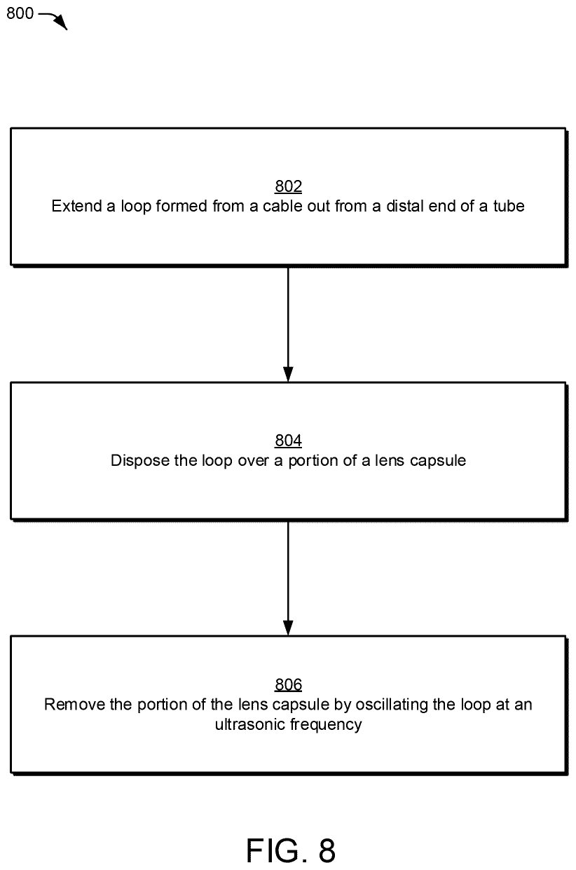

FIG. 8 illustrates an example method 800 for removing a portion of a lens capsule, according to embodiments described herein. At operation 802, the loop 210 formed from the cable 208 is extended out from the distal end 205 of the tube 204.

At operation 804, the loop 210 is disposed over a portion 222 of the lens capsule 112. At operation 806, the portion 222 of the lens capsule 112 is removed to form the circular hole 224 by oscillating the loop 210 at the ultrasonic frequency. In some embodiments, the loop 210 may be vibrated at a frequency between 10 kilohertz (kHz) to 60 kHz. Other frequencies may also be contemplated. In some embodiments, the ultrasonic frequency may be sufficient to disrupt the lens capsule portion in contact with the loop 210 to form the cut (e.g., through localized heat, mechanical scoring, abrasion, cavitation, cutting, etc.) In some embodiments, the loop 210 may be ultrasonically vibrated in a longitudinal direction, transverse direction, torsionally, etc. In some embodiments, the loop 210 may be vibrated using a combination of longitudinal, transverse, and/or torsional directions. In some embodiments, ends 208a and 208b may be moved in opposing longitudinal directions (for example, each may be attached to an opposing ultrasonic horn). In some embodiments, the heat and/or movement produced by the localized vibration may break down the capsule tissue along a controlled geometry to create a capsulorhexis.

The disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

What is claimed is:

1. A surgical instrument comprising:
a tube having a distal end and a proximal end;
a loop formed with a cable that is folded between a first end of the cable and a second end of the cable, wherein:
the cable is at least partially disposed in the tube, and
the first end of the cable is exposed from the proximal end of the tube; and
a horn, wherein:
the horn is configured to oscillate,
the first end of the cable is coupled to the horn, and
oscillation of the horn actuates the cable.

2. The surgical instrument of claim 1, wherein the horn is configured to oscillate at an ultrasonic resonance frequency.

3. The surgical instrument of claim 1, wherein the loop is extendable out from the tube at the distal end and retractable into the tube at the distal end.

4. The surgical instrument of claim 3, wherein an extension of the loop out from the tube at the distal end is configured to expand a shape of the loop from a compressed shape to a decompressed shape.

5. The surgical instrument of claim 1, wherein the loop includes at least one tooth.

6. The surgical instrument of claim 5, wherein the at least one tooth includes angled lateral edges or parallel lateral edges.

7. The surgical instrument of claim 5, wherein:
the loop includes at least one additional tooth;
the at least one tooth is oriented at a first angle relative to a cross-section of the loop; and
the at least one additional tooth is oriented at a second angle relative to the cross-section of the loop.

8. The surgical instrument of claim 1, wherein the loop is coating with a friction increasing material.

9. The surgical instrument of claim 1, wherein the loop includes a blade.

10. A method comprising:
extending a loop formed from a cable out from a distal end of a tube, the cable at least partially disposed in the tube;
disposing the loop over a portion of a lens capsule; and
removing the portion of the lens capsule by oscillating the loop at an ultrasonic frequency.

11. The method of claim 10, wherein the cable is coupled to an ultrasonic horn.

12. The method of claim 10, further comprising expanding a shape of the loop from a compressed shape to a decompressed shape.

13. The method of claim 10, wherein the loop includes at least one tooth.

14. The method of claim 13, wherein the at least one tooth includes at least one of angled lateral edges or parallel lateral edges.

15. The method of claim 10, wherein the loop includes at least one blade.
